(19) United States
(12) Patent Application Publication
(10) Pub. No.: US 2010/0292515 A1
(43) Pub. Date: Nov. 18, 2010

(54) Purification method of high-molecular-weight polyethylene glycol compound

(75) Inventors: Yuji Yamamoto, Kanagawa (JP); Hiroki Yoshioka, Kanagawa (JP)

Correspondence Address: SUGHRUE-265550, 2100 PENNSYLVANIA AVE. NW, WASHINGTON, DC, 20037-3213, US

(73) Assignee: NOF CORPORATION, Tokyo (JP)

(21) Appl. No.: 12/751,195

(22) Filed: Mar. 31, 2010

(30) Foreign Application Priority Data
Mar. 31, 2009 (JP) P2009-084773

(51) Int. Cl. C07C 43/178; C07C 43/11

(52) U.S. Cl. 568/607; 568/621

(57) ABSTRACT

An impurity derived from a high-molecular-weight polyethylene glycol compound is removed from a high-molecular-weight polyethylene glycol compound whose total average number of moles of ethylene oxide units added in the molecule is 220 to 4500. In a state where the high-molecular-weight polyethylene glycol compound is dissolved in at least one of water and an organic solvent selected from aromatic hydrocarbon solvents having 8 or less carbon atoms in total and ester compound solvents having 5 or less carbon atoms in total, the water and the organic solvent are mixed. The resulting mixture was separated into an organic layer and an aqueous layer, and the organic layer is separated from the aqueous layer.

### FIELD OF THE INVENTION

The present invention relates to a purification method of a high-molecular-weight polyethylene glycol compound. More specifically, the invention relates to a purification method of obtaining a high-molecular-weight activated polyethylene glycol compound to be used in pharmaceutical uses mainly including chemical modification of physiologically active proteins such as enzymes and the other drugs and chemical modification of liposomes, polymer micelles, and the like in drug delivery systems or a highly pure high-molecular-weight polyethylene glycol raw material useful as a starting material of the compound.

The invention is particularly suitable in pharmaceutical uses including modification of polypeptides, enzymes, antibodies, and other low-molecular-weight drugs, nucleic acid compounds including genes, oligonucleic acids, and the like, nucleic acid medicaments, and other physiologically active substances or application to drug delivery system carriers such as liposomes, polymer micelles, nanoparticles, and gel devices.

### BACKGROUND OF THE INVENTION

Recently, activated polyethylene glycols have been widely used as important carriers for drug delivery systems. As such activated polyethylene glycols for the purpose of the pharmaceutical uses, those containing little impurities have been required from the viewpoints of performance and safety of drugs to be produced by modifying them. Among the impurities in such activated polyethylene glycols, those having a large influence on the performance of drugs are polyethylene glycol impurities each having a molecular weight different from that of the objective compound, which may possibly change the in vivo pharmacokinetics and physical properties of the drugs. Particularly, in the case of a high-molecular-weight polyethylene glycol compound, such polyethylene glycol impurities are very difficult to remove by conventional technologies and hence result in a big problem.

For example, as mentioned in JP-A-11-335460, it is widely known that a polyethylene glycol impurity having hydroxyl groups at both terminals and having a molecular weight about twice that of the objective compound, which is derived from a small amount of water and is called a diol compound, is contained as an impurity in monomethoxypolyethylene glycol to be used as a raw material of many activated polyethylene glycols. The impurity results in a big problem at the time when the activated polyethylene glycol is applied to modification of drugs and the like. In the case where an activated polyethylene glycol is synthesized using such a raw material, hydroxyl groups positioned at both terminals of the polyethylene glycol impurity are activated as a result, and a polyethylene glycol impurity having two activated groups and having a larger molecular weight is formed as a by-product. When the activated polyethylene glycol having such a polyethylene glycol impurity is used for modification of a drug, as a result, drugs modified with polyethylene glycols different in molecular weight are contained and they have a large influence on the in vivo pharmacokinetics and physical properties of the drug, so that it becomes necessary to purify it at some stage(s).

However, the purification of the drug after the bonding of polyethylene glycol has a technical problem that the separation is difficult and, at the same time, a very big problem in cost that a drug yield is remarkably decreased. Accordingly, it is desirable to remove the polyethylene glycol impurity prior to the bonding to the drug.

For example, as shown in JP-A-8-165343, it is shown that an activated polyethylene glycol having a functional group having an amine is possible to separate by a chromatogram using an ion-exchange resin, and there is a method of purification at a stage of the activated polyethylene glycol prior to the reaction with a drug. However, such a purification method is limited to an application to a functional group having a charge which has affinity to the ion-exchange resin.

In consideration of generality, applicability to polyethylene glycol compounds having no ionic functional group is important. In particular, when development to many kinds thereof and industrial efficiency are considered, applicability to a polyethylene glycol compound having a hydroxyl group or a specific protective group, which is a precursor for the activated polyethylene glycol, is particularly a very big problem. Actually, with regard to an increase in purity of monomethoxypolyethylene glycol which is widely used as a raw material of activated polyethylene glycols, many methods have been reported.

With regard to conventional technologies, the following will describe the application to a high-molecular-weight polyethylene glycol compound having a molecular weight of about 20,000 or more which is a mainstream of current use particularly due to its high performance and the industrial applicability and versatility for actual performance in industrial scales as main points at issue.

One method is a method of obtaining a highly pure methoxypolyethylene glycol which is a polyethylene glycol raw material, by optimizing its synthetic method as shown in JP-A-11-335460 and US2006/0074200. In these examples, the influence of the water molecule causing the diol compound as an impurity having a higher molecular weight is suppressed to the minimum to suppress the formation of the diol compound by controlling water in the system in the ppm order in the ethylene oxide addition reaction using an alcohol compound as a starting material. The method is shown to be a method applicable to the high-molecular-weight methoxypolyethylene glycol having a molecular weight of 20,000 or more and is a method also excellent in industrial productivity. However, for producing a high-molecular-weight methoxypolyethylene glycol under the control of water in a reaction system in such an extremely low order of several ppm, a high level technology is required and introduction of a specialized expensive facility is required.

Moreover, in US2006/0074200, there is described a production method wherein an amount of water before EO addition is suppressed to such an extremely minute amount as 10 ppm or less. However, the formation of at least 2% or more of the diol is observed at the synthesis of the high-molecular-weight methoxypolyethylene glycol having a molecular weight of 20,000 or more and it is suggested that there is technically a certain limitation in the reduction of content of the diol compound by thoroughly removing water from such a polymerization system by any conventional technologies.

Another method for reducing such an impurity is a method of removing the diol compound as an impurity having a higher molecular weight from a high-molecular-weight methoxypolyethylene glycol having a terminal hydroxyl group by purification to reduce the impurity. As examples of representative experiments, there may be mentioned purification by dialysis in “Makromol. Chem., 189, 1809-1817 (1988) Leonard” and purification on a silica gel column in “J. Bioactive Compatible Polymers, 16, 206-220 (2001) Lapienis”. Thus, it is shown that it is possible to separate and remove the polyethylene glycol impurity in a small scale by these technologies but both cases are application to the purification of a polyethylene glycol compound having a relatively low molecular weight of about 5,000 or less, which corresponds to the average number of moles of ethylene glycol (oxyethylene group) added of about 110. There is not described the applicability to the high-molecular-weight polyethylene glycol having a molecular weight of 20,000 or more which is more difficult to separate.

It is a purification example in U.S. Pat. No. 5,298,410, wherein these technologies are further advanced and a possibility of practical use is extended. In this example, there is shown an experiment of isolation of methoxypolyethylene glycol containing little amount of the diol compound through a plurality of stages, wherein methoxypolyethylene glycol is modified with a dimethyltrityl group, a difference in polarity is amplified by chemical modification and fractionation is performed by a column chromatogram, and then the dimethyltrityl group of the corresponding fraction is eliminated. A similar technology is also described in JP-T-2008-514693, which is a technology that methoxypolyethylene glycol is modified with an acetic acid ester group or phthalic acid ester, a difference in polarity is also amplified by chemical modification and fractionation is performed by a column chromatogram, and then the group of the corresponding fraction is eliminated. It is shown that it is possible to carry out the technology in a larger scale and on the high-molecular-weight polyethylene glycol having a molecular weight of 20,000 or more.

### SUMMARY OF THE INVENTION

However, from the viewpoint that separation by a chromatogram is applied in these all examples, operations should be performed under a dilute condition of about 1 to 2% at most in these examples, much time is required for the separation, introduction of a large column apparatus is necessary, and a waste of a large amount of chromatogram gel is finally discharged, so that the examples contains many problems on industrial uses.

With regard to U.S. Pat. No. 5,298,410 and JP-T-2008-514693, purification efficiency is improved as compared with the conventional technologies, while there newly arise two problems that the steps are very complex and vexatious since the methyltrityl group or the acetic acid ester group, the phthalic acid ester group, or the like is once introduced by chemical modification, deprotection is performed after purification using it, and it is necessary to restore a hydroxyl group and also there is a possibility that an impurity having a new chemical species is formed since the chemical modification is performed during the steps. In particular, the latter is an extremely important problem, which may lead to complication of an impurity profile of an activated polyethylene glycol to be produced starting from the methoxypolyethylene glycol.

On the other hand, in WO2006/028745, there is shown an example where methoxypolyethylene glycol is allowed to act on an ion-exchange resin comprising a polycarboxylic acid to adsorb and remove the strongly interacting diol compound. This technology is shown to be an effective purification method also in the high-molecular-weight polyethylene glycol having a molecular weight of 20,000 or more. Furthermore, the technology does not use a column chromatogram and is constituted by simple steps of adsorption onto an ion-exchange resin and filtration, so that it is possible to avoid some problems of the column chromatogram as mentioned above. However, since such a purification method using an ion-exchange resin is a method of principally utilizing interaction and adsorption phenomenon to a solid surface similar to the above production method utilizing the column chromatogram, it is necessary to perform the purification treatment using a large amount of the resin under a dilute solution condition and the step has to be performed under such dilution that the concentration of methoxypolyethylene glycol in the step is about 1 to 2%, so that the method is not sufficiently satisfactory from the viewpoint of industrial productivity. Moreover, finally, a waste of a large amount of the ion-exchange resin is discharged and thus this method is also a purification method having a problem on industrial use.

From the above, at present, the method of removing a diol having a higher molecular weight from methoxypolyethylene glycol as a raw material for an activated polyethylene glycol still has problems on applicability and industrial practicability.

Moreover, on the other hand, as in Japanese Patent No. 3626494, in a branched polyethylene glycol typically obtained through a coupling reaction of two or more linear activated polyethylene glycols, the linear activated polyethylene glycol is used as a raw material and hence a polyethylene glycol impurity, which is one half in molecular weight as compared with the objective compound, is to be contained. In such a case, when a branched polyethylene glycol having an ionic group such as an amine group is obtained as a product, it is possible to separate it from the polyethylene glycol impurity different in molecular weight by a column chromatogram using an ion-exchange resin as in U.S. Pat. No. 5,932,462. However, such purification using an ion-exchange column chromatogram is not effective against the combination of a product having no ionic group and the impurity and thus is problematic on versatility. Furthermore, the introduction of a large column apparatus is necessary at the ion-exchange column chromatogram and also finally, a waste of a large amount of the ion-exchange resin is discharged, so that the purification also contains a problem on industrial applicability.

Incidentally, according to JP-A-2004-197077, a high-molecular-weight polyethylene glycol is obtained through a step of polymerizing ethylene oxide from a monovalent or polyvalent starting material having hydroxyl group(s) and a subsequent activation step.

As above, various high-molecular-weight polyethylene glycols for use in pharmaceutical uses all contain a polyethylene glycol impurity different in molecular weight depending on the production method and many problems exist on the removal thereof.

An object of the invention is to obtain a highly pure high-molecular-weight polyethylene glycol compound having a reduced content of the polyethylene glycol impurity different in molecular weight from the main component.

Also, an object of the invention is to provide a purification method which does not principally have a possibility of generation of new impurity species derived from polyethylene glycol, is industrially easily practicable, is also excellent in productivity, and does not form wastes such as gels and resins.

As a result of the extensive studies for solving the above problems, the present inventors have found a purification method of a high-molecular-weight polyethylene glycol compound wherein a specific extraction operation is performed in a system consisting of an organic solvent and an aqueous solution of a salt, which has a certain composition. A characteristic feature of the invention lies on a point that the invention provides a purification step which involves no chemical modification of the structure, is easily practicable in a large scale and industrially applicable, does not use any devices such as a large amount of carrier/adsorbent such as a resin or gel, ultrafiltration membrane, and the like, and also has a characteristic feature advantageous toward a subsequent chemical modification step, by using as the organic solvent a specific aromatic hydrocarbon solvent having an appropriate solubility to the high-molecular-weight polyethylene glycol compound or a specific organic solvent containing an ester compound solvent as a main component for extraction. Moreover, the invention has a useful characteristic feature in a point that it becomes possible to remove a polyethylene glycol impurity at a low-molecular-weight side by a combination of the specific extraction operation.

Namely, the invention is as shown below.

(1) A purification method through removing, from a high-molecular-weight polyethylene glycol compound whose total average number of moles of ethylene oxide units added in the molecule is 220 to 4500, a polyethylene glycol impurity different in molecular weight from the high-molecular-weight polyethylene glycol compound, which comprises:

(A) a mixing step of, in a state that the high-molecular-weight polyethylene glycol compound is dissolved in at least one of water and one or more organic solvents selected from the group consisting of aromatic hydrocarbon solvents having 8 or less carbon atoms in total and ester compound solvents having 5 or less carbon atoms in total, mixing the water and the organic solvent(s); and

(B) a separation step of separating the resulting mixture into an organic layer and an aqueous layer and separating the organic layer from the aqueous layer.

(2) The method according to the above (1), wherein the high-molecular-weight polyethylene glycol compound is represented by the general formula [1]:

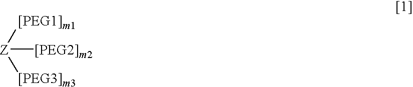

wherein Z is a divalent to octavalent bonding site having 30 or less atoms in total excluding hydrogen atom(s); PEG1, PEG2, and PEG3 are polyethylene glycol chains each having a different structure containing a bonding site and a terminal group from one another, and PEG1 and PEG2 are linear ones and PEG3 is branched one, respectively; m1, m2, and m3 represent the numbers of PEG1, PEG2, and PEG3 which bond to Z, respectively; and 0≦m≦1≦8, 0≦m2≦8, 0≦m3≦8, and 2≦m1+m2+m3≦8.

(3) The method according to the above (1) or the above (2), wherein an organic solvent is newly added to the aqueous layer separated in the separation step (B), and the mixing step (A) and the separation step (B) are repeated.

(4) The method according to the above (1) or the above (2), wherein water is newly added to the organic layer separated in the separation step (B), and the mixing step (A) and the separation step (B) are repeated.

(5) The method according to any one of the above (1) to the above (4), wherein the organic solvent is one or more solvents selected from the group consisting of xylene, toluene, benzene, methyl acetate, ethyl acetate, and butyl acetate.

(6) The method according to the above (5), wherein the organic solvent is toluene or ethyl acetate.

(7) The method according to any one of the above (1) to the above (6), wherein one or more additive solvents selected from the group consisting of hexane, cyclohexane, methylene chloride, chloroform, methanol, ethanol, isopropanol, tert-butanol, diethyl ether, methyl tert-butyl ether, tetrahydrofuran, N,N′-dimethylformamide, N,N′-dimethylform sulfoxide, and N,N′-dimethylacetamide are mixed into the organic solvent in an amount of 10% by mass.

(8) The method according to the above (7), wherein the additive solvent is one or more solvents selected from the group consisting of methanol and ethanol.

(9) The method according to any one of the above (1) to the above (8), wherein at least one of an organic salt and an inorganic salt is dissolved into the water.

(10) The method according to the above (9), wherein 3 to 20% by mass of an alkali metal inorganic salt or an alkali metal organic salt is dissolved into the water.

(11) The method according to any one of the above (1) to the above (10), wherein the mixing step (A) and the separation step (B) are carried out at 50 to 90° C.

(12) The method according to any one of the above (1) to the above (11), wherein the total amount of the organic solvent is 1 to 50 mass times the amount of the high-molecular-weight polyethylene glycol compound and the amount of water or the total amount of the water, the organic salt, and the inorganic salt is 0.1 to 50 mass times the amount of the high-molecular-weight polyethylene glycol compound.

(13) The method according to any one of the above (1) to the above (12), wherein the amount of the high-molecular-weight polyethylene glycol compound is 2 to 50 when the total amount of the organic solvent(s) and the water at the time of mixing is regarded as 100.

(14) The method according to any one of the above (1) to the above (13), wherein the total average number of moles of ethylene oxide units added in the molecule of the high-molecular-weight polyethylene glycol compound is 440 to 3500.

(15) The method according to any one of the above (2) to the above (14), wherein, in the general formula [1], m1 is 1, m2 is 1, and m3 is 0; and PEG1 is represented by the following general formula [2]:

—(CH₂CH₂O)ₙ₁-(A¹)ₐ-R¹ [2]

and PEG2 is represented by the following general formula

—(CH₂CH₂O)ₙ₂-(A²)_{b}-X² [3]

wherein R¹ is a hydrocarbon group having 1 to 7 carbon atoms or an acetal group having 4 to 9 carbon atoms; X² is a functional group or a protective group of a functional group and is different from R¹; n1 and n2 each is the average number of moles of ethylene oxide units added and n1+n2 is 220 or more and 4500 or less; A¹ and A² each independently is a divalent bonding site group having 30 or less carbon atoms and consisting of —CH₂—, —O—, —S—, —NH—, —CONH—, —NHCO—, —OCONH—, —NHOCO—, —COO—, —OCO—, —COS—, —SOC—, —S—S—, and a combination of groups selected from the group consisting of them, which does not contain —CH₂CH₂—O—; and a and b are the numbers of units of A¹ and A², respectively and each is 0 or 1.

(16) The method according to the above (15), wherein Z is —O—, X² is a hydroxyl group, a is 0, b is 1, and A² is —CH₂—CH₂—.

(17) The method according to the above (16), wherein R¹ is a methyl group.

(18) The method according to any one of the above (2) to the above (14), wherein, in the general formula [1], m1 is 2 or more and 7 or less, m2 is 1, and m3 is 0; and PEG1 is represented by the following general formula [2]:

—(CH₂CH₂O)ₙ₁-(A¹)ₐ-R¹ [2]

and PEG2 is represented by the following general formula [3]:

—(CH₂CH₂O)ₙ₂-(A²)_{b}-X² [3]

wherein R¹ is a hydrocarbon group having 1 to 7 carbon atoms or a functional group or a protective group of a functional group; X² is a functional group or a protective group of a functional group and is different from R¹; n1 and n2 each is the average number of moles of ethylene oxide units added and (n1×m1)+n2 is 220 or more and 4500 or less; A¹ and A² each independently is a divalent bonding site group having 30 or less carbon atoms and consisting of —CH₂—, —O—, —S—, —NH—, —CONH—, —NHCO—, —OCONH—, —NHOCO—, —COO—, —OCO—, —COS—, —SOC—, —S—S—, and a combination of groups selected from the group consisting of them, which does not contain —CH₂CH₂—O—; and a and b are the numbers of units of A¹ and A², respectively and each is 0 or 1.

(19) The method according to any one of the above (2) to the above (14), wherein, in the general formula [1], m1 is 0, m2 is 1, and m3 is 2 or more and 7 or less; and PEG2 is represented by the following general formula [3]:

—(CH₂CH₂O)ₙ₂-(A²)_{b}-X² [3]

and PEGS is represented by the following general formula [4]:

—(CH₂CH₂O)ₙ₃—Z′—[(CH₂CH₂O)ₙ₄-(A³)_{c}-R³]m₄ [4]

wherein R³ is a hydrocarbon group having 1 to 7 carbon atoms or a functional group or a protective group of a functional group; X² is a functional group or a protective group of a functional group and is different from R³; n2, n3, and n4 each is the average number of moles of ethylene oxide units added and n2+(n3+(n4×m4))×m3 is 220 or more and 4500 or less; A² and A³ each independently is a divalent bonding site group having 30 or less atoms in total excluding hydrogen atom(s) and consisting of —CH₂—, —O—, —S—, —NH—, —CONH—, —NHCO—, —OCONH—, —NHOCO—, —COO—, —OCO—, —COS—, —SOC—, —S—S—, and a combination of groups selected from the group consisting of them, which does not contain —CH₂CH₂—O—; Z′ is a divalent to nonavalent bonding site having 30 or less carbon atoms; m4 is the number of [(CH₂CH₂O)ₙ₄-(A³)_{c}-R³] bonded to Z′ and m4 is 1 or more and 8 or less; and b and c are the numbers of units of A² and A³, respectively and each is 0 or 1.

The method according to any one of the above (1) to the above (18), wherein the high-molecular-weight polyethylene glycol compound is collected from the organic layer.

(20) The method according to any one of the above (1) to the above (19), wherein the high-molecular-weight polyethylene glycol compound is collected from the aqueous layer.

(21) The method according to any one of the above (1) to the above (19), wherein the high-molecular-weight polyethylene glycol compound is collected from the organic layer.

(22) The method according to the above (21), wherein the high-molecular-weight polyethylene glycol compound is collected from the organic layer by a step including crystallization or solvent removal.

(23) The method according to the above (20), wherein the high-molecular-weight polyethylene glycol compound is collected from the aqueous layer by a step including any of spray-drying, drying, freeze-drying, extraction into an organic layer, and crystallization.

The invention provides a purification method of a highly pure high-molecular-weight polyethylene glycol for the purpose of pharmaceutical uses including modification of polypeptides, enzymes, antibodies, and other low-molecular-weight drugs, nucleic acid compounds including genes, oligonucleic acids, and the like, nucleic acid medicaments, and other physiologically active substances or modification to drug delivery system carriers such as liposomes, polymer micelles, nanoparticles, and gel devices. By applying the purification method, the removal of the polyethylene glycol impurities different in molecular weight in the high-molecular-weight polyethylene glycol compound can be performed by steps which are industrially easily practicable, also are excellent in productivity, and do not form wastes such as gels and resins.

An extraction operation of separation into an organic layer and an aqueous layer using polyethylene glycol as a solute is generally regarded as a method of separating polyethylene glycol and a substance largely different in polarity such as an ionic low-molecular weight substance and, before the invention, it is difficult to consider that such a high-molecular-weight polyethylene glycol compound is partitioned between an organic layer and an aqueous layer in a distinctly different ratio depending on the difference in molecular weight and the method is usable as a purification technology.

### BRIEF DESCRIPTION OF THE DRAWINGS

FIG. 1 It shows GPC chromatograms of fractions 1 to 4 obtained in Example 1.

FIG. 2 It shows GPC chromatograms of fractions 1 to 4 obtained in Example 10.

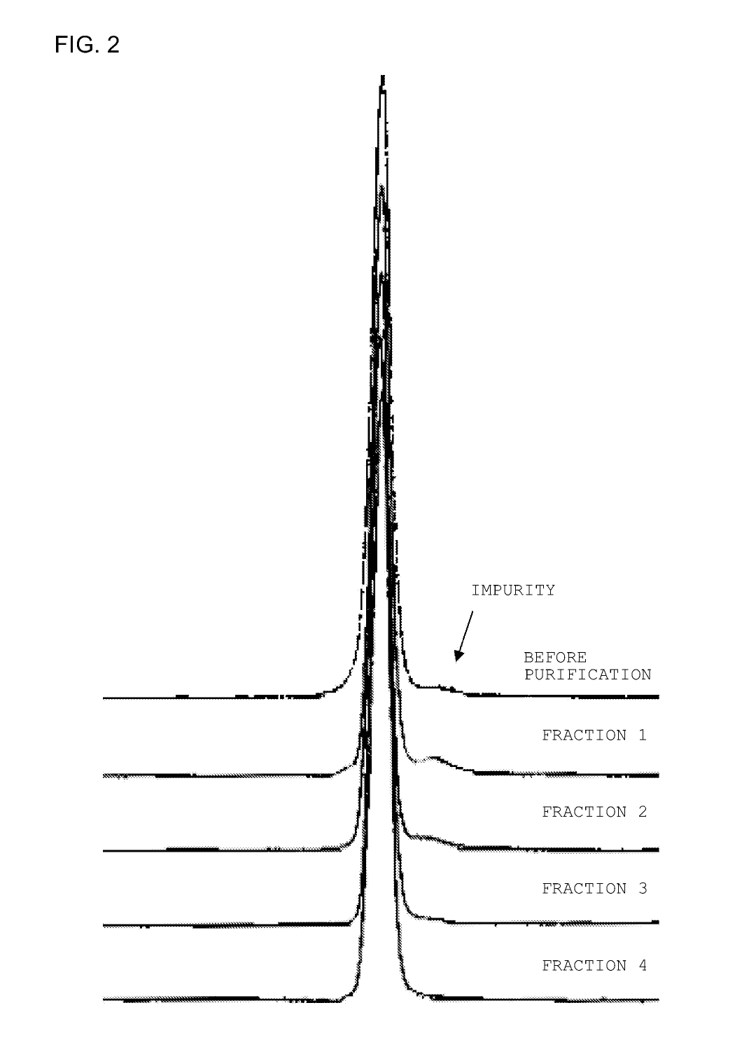

### DETAILED DESCRIPTION OF THE INVENTION

The invention relates to a purification method of a high-molecular-weight polyethylene glycol compound. More specifically, the invention relates to a purification method of obtaining a highly pure high-molecular-weight activated polyethylene glycol compound to be used in pharmaceutical uses mainly including chemical modification of physiologically active proteins such as enzymes and other drugs and chemical modification of drug carriers such as liposomes and polymer micelles, and surface modification of medical materials such as catheter or a highly pure high-molecular-weight polyethylene glycol raw material useful as a starting material of the compound.

The activated polyethylene glycol of the invention is a polyethylene glycol compound having a functional group capable of reacting with the other molecule on at least one terminal. The activated polyethylene glycol is to be used in pharmaceutical uses mainly including chemical modification of physiologically active proteins such as enzymes and other drugs and chemical modification of drug carriers such as liposomes and polymer micelles and includes one having not only a linear polyethylene glycol structure but also a branched polyethylene glycol structure.

The high-molecular-weight polyethylene glycol compound to be purified by the invention is the activated polyethylene glycol as mentioned above and a polyethylene glycol compound having a high molecular weight for the purpose of being used as a starting material thereof. The lower limit of the average number of moles of the ethylene oxide units added in the molecule of the high-molecular-weight polyethylene glycol compound is 220, preferably 440, and more preferably 660 and the upper limit is 4500, preferably 3500, more preferably 2500, and most preferably 2000. Moreover, preferably, the structure is represented by the following general formula [1]:

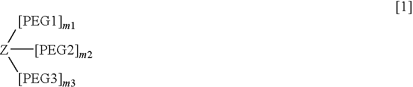

wherein Z is a divalent to octavalent bonding site and desirably does not have a large influence on dissolution properties of polyethylene glycol, preferably a divalent to octavalent bonding site having 30 or less carbon atoms, more preferably a divalent to octavalent bonding site group having 30 or less carbon atoms containing at least one bonding group of any one of —O—, —S—, —NH—, —CONH—, —NHCO—, —OCONH—, —NHOCO—, —COO—, —OCO—, —COS—, —SOC—, and —S—S—, and most preferably a bonding site having 30 or less carbon atoms containing at least one —O—. For example, specific examples of divalent, trivalent, and tetravalent bonding sites include the following structures but are not limited thereto.

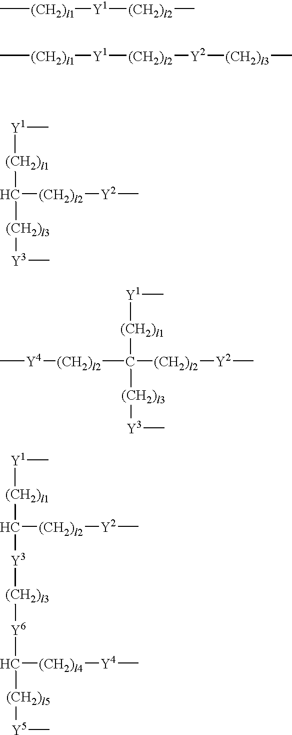

Here, 11, 12, 13, 14, and 15 each independently is an integer of 0 or more and the sum of respective ones in each molecule is 30 or less. Y1, Y2, Y3, Y4, Y5, and Y6 each independently is a bonding group and selected from —O—, —S—, —NH—, —CONH—, —NHCO—, —OCONH—, —NHOCO—, —COO—, —OCO—, —COS—, —SOC—, and —S—S—.

PEG1, PEG2, and PEG3 are polyethylene glycol segments each having a different structure containing a bonding site and a terminal group and PEG1 and PEG2 are linear chain ones and PEG3 is branched one having one or more branching points in the structure, respectively. m1, m2, and m3 each represents the number of polyethylene glycol segments and 0≦m1≦8, 0≦m2≦8, 0≦m3≦8, and 2≦m1+m2+m3≦8.

The general formula [1] is further preferably a linear polyethylene glycol compound wherein m1 is l, m2 is 1, m3 is 0, PEG1 is represented by the general formula [2]:

—(CH₂CH₂O)ₙ₁-(A¹)ₐ—R¹ [2]

PEG2 is represented by the general formula [3]:

—(CH₂CH₂O)ₙ₂-(A²)_{b}-X² [3]

and Z is divalent one.

Alternatively, it is a branched polyethylene glycol compound wherein, in the general formula [1], m1 is 2 to 7, m2 is l, m3 is 0, PEG1 is represented by the general formula [2], PEG2 is represented by the general formula [3], and Z is trivalent or higher valent one.

Alternatively, it is a multibranched polyethylene glycol compound wherein, in the general formula [1], m1 is 0, m2 is l, m3 is 2 to 7, PEG2 is represented by the general formula [3], PEG3 is represented by the general formula [4]:

—(CH₂CH₂O)ₙ₃—Z′—[(CH₂CH₂O)ₙ₄-(A³)_{c}-R³]m₄ [4]

Z is trivalent or higher valent one, and a branching point is also present in PEG3.

Here, R¹ represents a terminal-constituting element of the linear polyethylene glycol corresponding to PEG, R³ represents a terminal-constituting element of the branched polyethylene glycol corresponding to PEG3, and X² represents a terminal-constituting element of the linear polyethylene glycol corresponding to PEG2, which is different from R¹ and R³. A¹, A², and A³ each separately is a divalent bonding site group. n1, n2, n3, and n4 each is the average number of moles of the ethylene oxide units added in each polyethylene glycol segment. Z is a divalent to octavalent bonding site and Z′ is a divalent to nonavalent bonding site, which are independent from each other. m4 is the number of polyethylene glycol segment(s) at the terminal side, which bonds to the bonding site Z′ of PEG3.

More specifically, R¹ or R³ is a capping group, a functional group, or a protective group of a functional group. The capping group is desirably a group which does not generate any remarkable surface activity in the combination with the amphipathic polyethylene glycol moiety from the viewpoints of easiness and necessary time at the layer separation in the extraction step, and preferably a hydrocarbon group having 1 to 7 carbon atoms or an acetal group having 4 to 9 carbon atoms. The hydrocarbon group having 1 to 7 carbon atoms includes alkyl groups such as a methyl group, an ethyl group, a propyl group, an isopropyl group, a butyl group, an isobutyl group, a sec-butyl group, a tert-butyl group, a pentyl group, an isopentyl group, a hexyl group, an isohexyl group, a heptyl group, and an isoheptyl group, a phenyl group, and a benzyl group. The acetal group having 4 to 9 carbon atoms includes a dimethoxyethane group, a dimethoxypropane group, a dimethoxybutane group, a dimethoxypentane group, a dimethoxyhexane group, a dimethoxyheptane group, a diethoxyethane group, a diethoxypropane group, a diethoxybutane group, and the like. In consideration of the usefulness and stability in the chemical modification after purification, the function and performance for the purpose of pharmaceutical uses including modification of drugs, nucleic acids, and drug delivery system carriers, and easiness of the layer separation in the extraction operation, preferably, the hydrocarbon group is a methyl group, an ethyl group, a tert-butyl group, or a benzyl group and the acetal group is a diethoxypropane group or a diethoxybutane group, and most preferred is a methyl group. The functional group is not limited but, in consideration of the stability of the functional group, is preferably an amino group, a carboxyl group, a hydroxyl group, a thiol group, a hydrazine group, a hydrazide group, an acetyl group, an azide group, or an oxyamine group, and preferred is a hydroxyl group. The protective group of the functional group is also not particularly limited but is preferably a protective group of an amino group, a carboxyl group, a hydroxyl group, a thiol group, a hydrazine group, a hydrazide group, an acetyl group, an azide group, an oxyamine group or an aldehyde group, and preferred is a protective group of a hydroxyl group.

X² is a functional group or a protective group of a functional group and is not limited but, in consideration of the stability of the functional group, is preferably an amino group, a carboxyl group, a hydroxyl group, or thiol group, a hydrazine group, a hydrazide group, an acetyl group, an azide group, an oxyamine group, or an protective group thereof or an protective group of an aldehyde group, and preferred is a hydroxyl group or a protective group of a hydroxyl group. However, it is a group different from R¹, and R³.

A¹ and A³ are linker sites between each polyethylene glycol segment and each of the terminal groups R¹ and R³, respectively, and each independently is a divalent bonding site group having 30 or less carbon atoms in total, consisting of a combination of groups selected from the group consisting of —CH₂—, —CONH—, —NHCO—, —OCONH—, —NHOCO—, —COO—, —OCO—, —COS—, —SOC—, —CH₂NH—, NHCH₂—, —S—, —S—S—, and —O—, which does not contain —CH₂CH₂—O—.

n1, n2, n3, and n4 are average numbers of moles of the ethylene oxide unit added in each polyethylene glycol segment, respectively, provided that, in the relation of m1, m2, m3, and m4, the total average number of moles of the ethylene oxide unit added in the molecule lies between the lower limit and the upper limit defined in the above. Namely, individually, n1+n2 lies between the lower limit and the upper limit in the case of m1=1, m2=1, and m3=0 in the general formula [1]; (n1×m1)+n2 lies between them in the case of m1=2 to 7, m2=1, and m3=0; and n2+(n3+(n4×m4))×m3 lies between them in the case of m1=0, m2=1, and m3=2 to 7. More preferably, the total average number of moles of the ethylene oxide unit added in the part of [PEG1]ₘ₁ in the case of m1=1, m2=1, and m3=0 or m1=2 to 7, m2=1, and m3=0, i.e., n1 or n1×m1 is larger than the lower limit of the total average number of moles of the ethylene oxide unit added as defined in the above. Similarly, the total average number of moles of the ethylene oxide unit added in the part of [PEG3]ₘ₃ in the case of m1=0, m2=1, and m3=2 to 7, i.e., (n3+(n4×m4))×m3 is larger than the lower limit of the total average number of moles of the ethylene oxide unit added as defined in the above.

In the invention, the high-molecular-weight polyethylene glycol compound represented by the above general formula [1] is obtained via a step of polymerizing ethylene oxide from a monovalent or polyvalent starting material having hydroxyl group(s) and a subsequent activation step as in JP-A-2004-197077 or is obtained typically via a coupling reaction of two or more linear polyethylene glycols and an activation step as in Japanese Patent No. 3626494. Moreover, the polyethylene glycol impurities which are to be removed in the invention and contained in the high-molecular-weight polyethylene glycol compound are not particularly limited except that they have a molecular weight different from that of the high-molecular-weight polyethylene glycol compound as a main component. However, in consideration of the synthetic routes as mentioned above, examples include a polyethylene glycol compound having both hydroxyl group terminals called a diol compound originated from the water contained in the starting substance at the polymerization reaction, a reactant generated by a side reaction between polyethylene glycol compounds themselves in the activation step, a tailing component toward a low-molecular-weight side derived from a stopping reaction or heterogeneity of stirring caused by a viscosity increase during the polymerization, a residual group of a polyethylene glycol compound originated from an unreacted product of the coupling reaction of polyethylene glycol compounds themselves, decomposition products generated in individual reaction steps including activation, and the like. The polyethylene glycol impurity diol compound contained in the above high-molecular-weight polyethylene glycol compound has a molecular weight about twice that of the high-molecular-weight polyethylene glycol compound in the case of using a monofunctional low-molecular-weight compound as a starting material of the polymerization as a typical example but, in the case of using a trifunctional or higher functional low-molecular-weight compound or a polyethylene glycol compound as a starting material of the polymerization, there is a case where the diol compound has a molecular weight lower than that of the high-molecular-weight polyethylene glycol compound.

The extraction step in the invention is a general operation without particular limitation and typically includes a step of mixing, by stirring, shaking, or the like, a mixed solvent consisting of an organic solvent and water or an aqueous solution of a salt and containing the high-molecular-weight polyethylene glycol compound dissolved therein, and separating the solvent into an organic layer and an aqueous layer by allowing it to stand for a certain period of time. Here, the organic layer and the aqueous layer after the layer separation contain the above organic solvent and the aqueous solution of the salt, respectively, as a main component but the composition is not necessarily completely coincident before and after the extraction step and the layers contains the above high-molecular-weight polyethylene glycol compound, impurities, the other solvent components, and the like. Moreover, in the extraction step, the high-molecular-weight polyethylene glycol compound may have been dissolved in a mixed solvent system consisting of the above organic solvent and the aqueous solution of the salt beforehand and it is not indicated that the compound is not dissolved in any of the organic solvent, water, or the aqueous solution of the salt in a step prior to the step but, in view of simplification of the step, it is preferable that the compound is dissolved in either of the above organic solvent or an organic solvent component constituting the same or in water or an aqueous solution of the above salt. The time for the mixing and layer separation during the step is not particularly limited but is preferably between 1 minute to 12 hours, and more preferably 10 minutes to 3 hours. Moreover, the atmosphere for performing the extraction operation is not particularly limited but, for the purpose of suppressing undesirable oxidation on the high-molecular-weight polyethylene glycol compound to the minimum, the operation is typically performed in the presence of an inert gas such as nitrogen. Moreover, in the case of purifying the high-molecular-weight polyethylene glycol compound having a structure or a functional group especially easily oxidized, an antioxidant or a reducing agent can be contained in the system. Furthermore, the apparatus is not particularly limited but the operation can be also performed in a pressure vessel in consideration of the operation under nitrogen and in a tightly closed state which hardly generates oxidation deterioration. Also, similarly, in the case where the high-molecular-weight polyethylene glycol compound having a structure or a functional group unstable in a specific pH region, pH in the system can be controlled to an appropriate range by adding a buffer solution or an acid or alkali.

The organic solvent for use in the extraction operation in the invention is an organic solvent selected from aromatic hydrocarbon solvents having 8 or less carbon atoms in total and ester compound solvents having 5 or less carbon atoms in total or a mixture thereof. From the viewpoint of purification efficiency of the high-molecular-weight polyethylene glycol compound as an objective of the invention, the organic solvent is preferably one or more solvents selected from xylene, toluene, benzene, methyl acetate, ethyl acetate, and butyl acetate and may be a mixture thereof, is more preferably toluene or ethyl acetate or may be a mixture thereof, and is most preferably toluene.

The reason for the use of the above organic solvent includes a property that the solvent does not have an excessive affinity to the high-molecular-weight polyethylene glycol compound to be used in the invention, has an appropriate solubility, and the layer separation is well performed since the solvent is not dissolved in water. Owing to such a property, an effective purification is possible under a condition where the solubility of the polyethylene glycol impurities different in molecular weight is different from that of the high-molecular-weight polyethylene glycol compound. Moreover, the use of such a solvent having an appropriate solubility to the high-molecular-weight polyethylene glycol compound is an effective property for isolating the above high-molecular-weight polyethylene glycol compound through crystallization by cooling or addition of a poor solvent after the extraction step. Moreover, another common characteristic property that the solvent is volatile also connects with an advantage in the following treatment step which presupposes isolation, the advantage being that the solvent removal is easily possible. Furthermore, the property of good separation from water is an advantage in the extraction step that the property not only contributes to improvement in purification efficiency and yield and shortening of the time required for the layer separation but also easily enables minimization of influence of water that is an obstacle at the subsequent activation reaction of the high-molecular-weight polyethylene glycol compound. In the case where particularly an aromatic hydrocarbon solvent such as toluene or benzene is used as the above organic solvent, it is possible to perform azeotropic removal of water at the time of concentration and isolation by the solvent removal and it is possible to reduce the amount of water in the obtained high-molecular-weight polyethylene glycol compound to a lower level.

As above, in the invention, the extraction operation using a specific aromatic hydrocarbon solvent having an extremely multilateral advantage or an ester compound solvent as an organic solvent is one significant characteristic feature from the viewpoint of purification of a high-molecular-weight activated polyethylene glycol compound to be used in pharmaceutical uses or a high-molecular-weight polyethylene glycol raw material as an origin thereof. In addition to the above organic solvent, for the purpose of controlling a layer separation rate and yield, it is possible to contain, in the system, an additive component consisting of an organic solvent defined in the following.

The above other additive organic solvent is not particularly limited but generally includes hydrocarbons including hexane and cyclohexane, chlorinated hydrocarbons such as methylene chloride and chloroform, alcohols such as methanol, ethanol, isopropanol, and tert-butanol, ethers such as diethyl ether and methyl tert-butyl ether, cyclic ethers such as tetrahydrofuran, and also N,N′-dimethylformamide, N,N′-dimethylform sulfoxide, N,N′-dimethylacetamide, and the like. In order to increase purification efficiency and perform the layer separation efficiently for a short time, it is particularly effective to add an alcohol such as methanol, ethanol, isopropanol, or tert-butanol, preferably methanol or ethanol. The amount of the additive component to be added is 10% by mass or less, preferably 5% by mass or less based on the organic solvent (the total amount of the organic solvent is regarded as 100% by mass).

As the aqueous solution of the salt to be used in the invention, an aqueous solution of an inorganic salt or an organic salt is used. Here, the inorganic salt or the organic salt is not particularly limited but is preferably an alkali metal salt, more preferably an alkali metal halogen salt, and most preferably sodium chloride. The salt concentration of the aqueous solution is not particularly limited but is preferably 3 to 20% by mass and more preferably 5 to 15% by mass in consideration of the purification effect and yield on the high-molecular-weight polyethylene glycol compound as a target in the invention since the transferring ratio of the high-molecular-weight polyethylene glycol compound into the organic layer increases with an increase in the salt concentration of the aqueous solution of the salt.

The amounts of the organic solvent and the aqueous solution of the salt to be used is not particularly limited but the purification efficiency and yield and the productivity are determined with balancing the amounts of both. When the fact is considered, preferably, the amount of the organic solvent is 1 to 50 mass times that of the above high-molecular-weight polyethylene glycol compound and the amount of water or the aqueous solution of the salt is 0.1 to 50 mass times that of the high-molecular-weight polyethylene glycol compound and more preferably, the amounts of both the organic solvent and the aqueous solution of the salt are 5 to 20 mass times that of the high-molecular-weight polyethylene glycol compound.

Moreover, in the extraction system of the invention, since not the purification by adsorption onto a two-dimensional surface but the purification utilizing a difference of solubility into each solvent component between the high-molecular-weight polyethylene glycol and the polyethylene glycol impurities is performed, it is possible to perform an operation in a region where the concentration of the high-molecular-weight polyethylene glycol relative to the above organic solvent and the aqueous solution of the salt is relatively high. In this case, in consideration of a relation between purification efficiency and yield, with regard to the concentration of the high-molecular-weight polyethylene glycol relative to the total amount of the whole organic solvent and water (further a salt if present), the amount of the high-molecular-weight polyethylene glycol compound is preferably 2 to 50, more preferably 3 to 30, and most preferably 5 to 20 when the total mass of the organic solvent and the aqueous solvent of the salt is regarded as 100 in the extraction system.

The temperature for performing the extraction operation is not particularly limited but, since the transferring ratio of the polyethylene glycol compound to the organic layer increases with an elevation of temperature of the system, the temperature is preferably 40 to 90° C., more preferably 45 to 80° C., and most preferably 50 to 70° C. when the purification effect and yield on the high-molecular-weight polyethylene glycol compound as a target in the invention is considered.

The content of the polyethylene glycol impurities different in molecular weight may be typically determined by analysis by gel permeation chromatography (GPC) capable of measuring molecular weight distribution of a polymer. In the invention, the measurement was carried out with using SHODEX GPC SYSTEM-11 as a GPC system and SHODEX RIX8 as a differential refractometer that is a detector, connecting three columns of SHODEX KF801L, KF803L, and KF804L (φ 8 mm×300 mm) in series as GPC columns, controlling the temperature of the column oven to 40° C., using tetrahydrofuran as an eluent, and controlling the flow rate to 1 ml/minute, the concentration of a sample to 0.1% by mass, and injection volume to 0.1 ml. As a calibration curve, there is used one prepared with using ethylene glycol, diethylene glycol, triethylene glycol manufactured by Kanto Chemical Co., Inc., and Polymer Standards for GPC manufactured by Polymer Laboratory, which are polyethylene glycols or polyethylene oxides each having a molecular weight of 600 to 70000. For data analysis, BORWIN GPC calculation program was used. With regard to the content of the polyethylene glycol impurity different in molecular weight, a peak area was sectioned with a straight line vertically drawn from a minimum point between peaks of the impurity and the main peak in a chromatogram obtained by the RI detector, a ratio of a peak area having an elution time faster than it, i.e., at a higher molecular weight side relative to the total area or a ratio of a peak area having an elution time slower than it, i.e., at a lower molecular weight side relative to the total area was calculated, the ratio being regarded as the content of each polyethylene glycol impurity having a different molecular weight. In the case where the peak of an impurity is extremely small or is not sharp and hence a distinct minimum point was not obtained, instead of the point, the peak area was sectioned with a straight line vertically drawn from an infection point of the chromatogram and the ratio was calculated in a similar manner. It is also possible to determine the content of the polyethylene glycol impurities different in molecular weight by another analytical means suitable for determining molecular weight distribution, such as a time of flight mass spectrometry apparatus (TOF-MS).

The treatment step after the extraction step in the invention is not particularly limited but, in the case of collecting the organic layer, typically, the high-molecular-weight polyethylene glycol can be isolated via crystallization operated by cooling the separated organic layer or adding a hydrocarbon such as hexane or cyclohexane, a higher alcohol such as isopropanol, or an ether such as diethyl ether or methyl tert-butyl ether as a poor solvent and following drying. Moreover, it is also possible to isolate the high-molecular-weight polyethylene glycol by removing the organic solvent system through solvent removal and drying and solidifying it. Furthermore, when the organic solvent used is not inhibit the following reaction, it is possible to use the organic layer containing the high-molecular-weight polyethylene glycol as it is in the activation reaction without these operations of crystallization and solvent removal. In the case where a strict control of the water content is necessary prior to these operations of isolation, reaction, and the like, additionally, the organic layer containing the above high-molecular-weight polyethylene glycol or the solution of the high-molecular-weight polyethylene glycol derived from the layer can be dehydrated typically using a dehydrating agent such as magnesium sulfate or sodium sulfate or, in the case where an organic solvent such as toluene or benzene is a main component, by azeotropic treatment. In the case of collecting the aqueous layer, the high-molecular-weight polyethylene glycol can be collected by spray-drying or freeze-drying without further treatment, or by a step including any of concentration, crystallization, drying, and the like via extraction into the organic layer.

### EXAMPLES

The following will explain the invention in detail with reference to Examples.

In Examples 1 to 9, a polyethylene glycol impurity to be removed from high-molecular-weight polyethylene glycol compound is an impurity originated from a diol compound, which has a molecular weight about twice that of the objective compound.

### Example 1

In a 300 mL four-neck flask fitted with a mechanical stirring apparatus, a Dimroth condenser, a thermometer, and a nitrogen-introducing tube were placed 10 g of methoxypolyethylene glycol represented by the formula [4] (molecular weight: 30,000, amount of high-molecular-weight impurity: 2.81%) and 100 g of toluene, which were then dissolved at 50° C. under nitrogen with stirring using a mantle heater. Thereto was added 100 g of a 10% by mass aqueous sodium chloride solution, and the whole was slowly stirred and heated to 68° C. After the temperature reached 68° C., the solution was stirred for 30 minutes and, after stirring was stopped, was allowed to stand at the same temperature for 10 minutes to effect layer separation. The organic layer as the separated upper layer was collected in a 300 mL eggplant-shape flask using a pipette. The organic layer containing toluene as a main component was concentrated at 80° C. to 20 g on an evaporator and, after the concentrate was cooled to 25° C. with stirring using a magnetic stirrer, 20 g of hexane was added thereto to precipitate crystals. The slurry was stirred for 30 minutes and filtrated and, after the residue was washed with 20 g of hexane, drying was performed under vacuum to collect a fraction 1 (2.7 g). Subsequently, 100 g of toluene was added to the remaining aqueous layer and the whole was slowly stirred and heated to 68° C. After the temperature reached 68° C., the solution was stirred for 30 minutes and then allowed to stand for 10 minutes. Thereafter, as in the case of the fraction 1, the collection of the toluene layer, concentration, crystallization with hexane, and drying were performed to collect a fraction 2 (2.4 g). In the following, similar operations were repeated and a fraction 3 (2.0 g) and a fraction 4 (1.0 g) were collected.

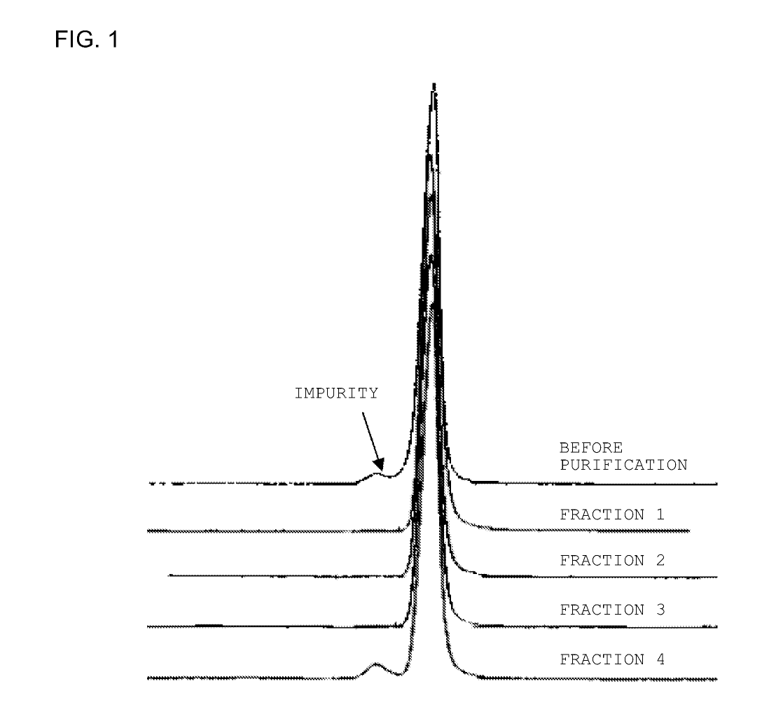

FIG. 1 shows GPC chromatograms of the obtained fractions 1 to 4. As shown in the figure, a line was vertically drawn from a minimum point between the elution peaks of the diol compound and methoxypolyethylene glycol toward a base line and the peak areas were assigned to the diol compound and methoxypolyethylene glycol. As a result, the respective amounts of the high-molecular-weight impurity in samples 1, 2, 3, and 4 were 0.39%, 0.46%, 0.55%, and 1.65%.

CH₃O—(CH₂CH₂O)ₙ—H [4]

### Example 2

In a 300 mL four-neck flask fitted with a mechanical stirring apparatus, a Dimroth condenser, a thermometer, and a nitrogen-introducing tube were placed 10 g of methoxypolyethylene glycol (molecular weight: 40,000, amount of high-molecular-weight impurity: 2.80%) and 100 g of toluene, which were then dissolved at 50° C. under nitrogen with stirring using a mantle heater. Thereto was added 50 g of a 10% by mass aqueous sodium chloride solution, and the whole was slowly stirred and heated to 68° C. After the temperature reached 68° C., the solution was stirred for 30 minutes and, after stirring was stopped, was allowed to stand at the same temperature for 10 minutes to effect layer separation. The organic layer as the separated upper layer was collected in a 300 mL eggplant-shape flask using a pipette. The organic layer containing toluene as a main component was concentrated at 80° C. to 20 g on an evaporator and, after the concentrate was cooled to 25° C. with stirring using a magnetic stirrer, 20 g of hexane was added thereto to precipitate crystals. The slurry was stirred for 30 minutes and filtrated and, after the residue was washed with 20 g of hexane, drying was performed under vacuum to collect a fraction 1 (2.0 g). Subsequently, 100 g of toluene was added to the remaining aqueous layer and the whole was slowly stirred and heated to 68° C. After the temperature reached 68° C., the solution was stirred for 30 minutes and then allowed to stand for 10 minutes. Thereafter, as in the case of the fraction 1, the collection of the toluene layer, concentration, crystallization with hexane, and drying were performed to collect a fraction 2 (1.0 g). In the following, similar operations were repeated and a fraction 3 (1.0 g) was collected.

The amounts of the high-molecular-weight impurity in the obtained fractions 1 to 3 were 0.42%, 0.17%, and 0.55%.

### Example 3

In a 100 mL four-neck flask fitted with a mechanical stirring apparatus, a Dimroth condenser, a thermometer, and a nitrogen-introducing tube were placed 10 g of methoxypolyethylene glycol (molecular weight: 40,000, amount of high-molecular-weight impurity: 2.80%) and 30 g of toluene, which were then dissolved at 50° C. under nitrogen with stirring using a mantle heater. Thereto was added 30 g of a 10% by mass aqueous sodium chloride solution, and the whole was slowly stirred and heated to 68° C. After the temperature reached 68° C., the solution was stirred for 30 minutes and, after stirring was stopped, was allowed to stand at the same temperature for 20 minutes to effect layer separation. The organic layer as the separated upper layer was collected in a 300 mL eggplant-shape flask using a pipette. The organic layer containing toluene as a main component was concentrated at 80° C. to 20 g on an evaporator and, after the concentrate was cooled to 25° C. with stirring using a magnetic stirrer, 20 g of hexane was added thereto to precipitate crystals. The slurry was stirred for 30 minutes and filtrated and, after the residue was washed with 20 g of hexane, drying was performed under vacuum to collect a fraction 1 (3.5 g). Subsequently, 30 g of toluene was added to the remaining aqueous layer and the whole was slowly stirred and heated to 68° C. After the temperature reached 68° C., the solution was stirred for 30 minutes and then allowed to stand for 10 minutes. Thereafter, as in the case of the fraction 1, the collection of the toluene layer, concentration, crystallization with hexane, and drying were performed to collect a fraction 2 (1.8 g). In the following, similar operations were repeated and a fraction 3 (0.8 g) was collected.

The amounts of the high-molecular-weight impurity in the obtained fractions 1 to 3 were 0.68%, 0.37%, and 0.39%.

### Example 4

In a 200 mL four-neck flask fitted with a mechanical stirring apparatus, a Dimroth condenser, a thermometer, and a nitrogen-introducing tube were placed 10 g of methoxypolyethylene glycol (molecular weight: 30,000, amount of high-molecular-weight impurity: 2.81%), 25 g of toluene, and 25 g of ethyl acetate, which were then dissolved at 50° C. under nitrogen with stirring using a mantle heater. Thereto was added 50 g of a 15% by mass aqueous sodium chloride solution, and the whole was slowly stirred and heated to 53° C. After the temperature reached 53° C., the solution was stirred for 30 minutes and, after stirring was stopped, was allowed to stand at the same temperature for 30 minutes to effect layer separation. The organic layer as the separated upper layer was collected in a 300 mL eggplant-shape flask using a pipette. The organic layer containing toluene as a main component was concentrated at 80° C. to 20 g on an evaporator and, after the concentrate was cooled to 25° C. with stirring using a magnetic stirrer, 20 g of hexane was added thereto to precipitate crystals. The slurry was stirred for 30 minutes and filtrated and, after the residue was washed with 20 g of hexane, drying was performed under vacuum to collect a fraction 1 (1.0 g). Subsequently, 25 g of toluene and 25 g of ethyl acetate were added to the remaining aqueous layer and the whole was slowly stirred and heated to 55° C. After the temperature reached 55° C., the solution was stirred for 30 minutes and then allowed to stand for 30 minutes. Thereafter, as in the case of the fraction 1, the collection of the toluene layer, concentration, crystallization with hexane, and drying were performed to collect a fraction 2 (6.6 g).

The amounts of the high-molecular-weight impurity in the obtained fractions 1 to 2 were 0.46% and 2.08%.

### Example 5

In a 200 mL four-neck flask fitted with a mechanical stirring apparatus, a Dimroth condenser, a thermometer, and a nitrogen-introducing tube were placed 10 g of α-diethoxypropanoxy-ω-methyl-polyethylene glycol represented by the formula [5] (molecular weight: 30,000, amount of high-molecular-weight impurity: 3.26%) and 50 g of ethyl acetate, which were then dissolved at 50° C. under nitrogen with stirring using a mantle heater. Thereto was added 50 g of a 13% by mass aqueous sodium chloride solution, and the whole was slowly stirred and heated to 54° C. After the temperature reached 54° C., the solution was stirred for 30 minutes and, after stirring was stopped, was allowed to stand at the same temperature for 30 minutes to effect layer separation. The organic layer as the separated upper layer was collected in a 300 mL eggplant-shape flask using a pipette. The organic layer containing toluene as a main component was concentrated at 80° C. to 20 g on an evaporator and, after the concentrate was cooled to 25° C. with stirring using a magnetic stirrer, 20 g of hexane was added thereto to precipitate crystals. The slurry was stirred for 30 minutes and filtrated and, after the residue was washed with 20 g of hexane, drying was performed under vacuum to collect a fraction 1 (2.5 g).

The amount of the high-molecular-weight impurity in the obtained fraction 1 was 0.33%.

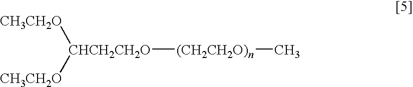

### Example 6

In a 200 mL four-neck flask fitted with a mechanical stirring apparatus, a Dimroth condenser, a thermometer, and a nitrogen-introducing tube were placed 10 g of α-benzyloxypolyethylene glycol (molecular weight: 30,000, amount of high-molecular-weight impurity: 3.29%) represented by the formula [6] and 70 g of toluene, which were then dissolved at 50° C. under nitrogen with stirring using a mantle heater. Thereto was added 70 g of a 10% by mass aqueous sodium chloride solution, and the whole was slowly stirred and heated to 68° C. After the temperature reached 68° C., the solution was stirred for 30 minutes and, after stirring was stopped, was allowed to stand at the same temperature for 30 minutes to effect layer separation. The organic layer as the separated upper layer was collected in a 300 mL eggplant-shape flask using a pipette. The organic layer containing toluene as a main component was concentrated at 80° C. to 20 g on an evaporator and, after the concentrate was cooled to 25° C. with stirring using a magnetic stirrer, 20 g of hexane was added thereto to precipitate crystals. The slurry was stirred for 30 minutes and filtrated and, after the residue was washed with 20 g of hexane, drying was performed under vacuum to collect a fraction 1 (1.2 g). Subsequently, 66.5 g of toluene and 3.5 g of ethanol were added to the remaining aqueous layer and the whole was slowly stirred and heated to 69° C. After the temperature reached 69° C., the solution was stirred for 30 minutes and then allowed to stand for 10 minutes. Thereafter, as in the case of the fraction 1, the collection of the toluene layer, concentration, crystallization with hexane, and drying were performed to collect a fraction 2 (2.6 g). In the following, similar operations were repeated and a fraction 3 (2.1 g) and a fraction 4 (1.2 g) were collected.

The amounts of the high-molecular-weight impurity in the obtained fractions 1 to 4 were 2.74%, 1.86%, 1.01%, and 0.38%.

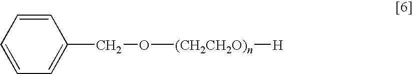

### Example 7

In a 3,000 mL four-neck flask fitted with a mechanical stirring apparatus, a Dimroth condenser, a thermometer, and a nitrogen-introducing tube were placed 200 g of methoxypolyethylene glycol (molecular weight: 40,000, amount of high-molecular-weight impurity: 2.80%) and 1,000 g of toluene, which were then dissolved at 50° C. under nitrogen with stirring using a mantle heater. Thereto was added 1,000 g of a 10% by mass aqueous sodium chloride solution, and the whole was slowly stirred and heated to 68° C. After the temperature reached 68° C., the solution was stirred for 10 minutes and, after stirring was stopped, was allowed to stand at the same temperature for 30 minutes to effect layer separation. The organic layer as the separated upper layer was collected in a 2,000 mL eggplant-shape flask placed in a bell jar under vacuum through a glass tube and a silicone tube. The toluene solution was concentrated at 80° C. to 500 g on an evaporator and, after 10 g of magnesium sulfate was charged thereto, dehydration was performed at 50° C. with stirring using a magnetic stirrer. After magnesium sulfate was removed by filtration, the solution was cooled to 25° C. and then hexane was added to precipitate crystals. The slurry was stirred for 30 minutes and filtrated and, after the residue was washed with 500 g of hexane, drying was performed under vacuum to collect a fraction 1 (108 g). Subsequently, 800 g of toluene was added to the remaining aqueous layer and the whole was slowly stirred and heated to 68° C. After the temperature reached 68° C., the solution was stirred for 30 minutes and then allowed to stand for 20 minutes. Thereafter, as in the case of the fraction 1, the collection of the toluene layer, concentration, dehydration, crystallization with hexane, and drying were performed to collect a fraction 2 (24 g).

The amounts of the high-molecular-weight impurity in the obtained fractions 1 to 2 were 1.01% and 0.58%.

### Example 8

In a 100 L stainless tightly closed vessel fitted with a mechanical stirring apparatus and a thermometer were placed 5 kg of methoxypolyethylene glycol (molecular weight: 40,000, amount of high-molecular-weight impurity: 2.80%) and 20 kg of toluene, which were then dissolved at 60° C. under nitrogen with stirring. Thereto was added 25 kg of a 10% by mass aqueous sodium chloride solution, and the whole was slowly stirred and heated to 70° C. After the temperature reached 70° C., the solution was stirred for 30 minutes and, after stirring was stopped, was allowed to stand at the same temperature for 3 hours to effect layer separation. The aqueous layer as the separated lower layer was first taken out from the bottom cock into a stainless vessel and the toluene layer as an upper layer was then collected from the bottom cock into another stainless vessel. The toluene solution was concentrated at 70° C. to 3.8 kg on an evaporator, the concentrate was again dissolved in 15 kg of toluene and, after 500 g of magnesium sulfate was charged, dehydration was performed at 60° C. with stirring. After magnesium sulfate was removed by filtration, the solution was cooled to 25° C. and then 5 kg of hexane was added thereto to precipitate crystals. The slurry was stirred for 30 minutes and filtrated and, after the residue was washed with 8 kg of hexane, drying was performed under vacuum to collect a fraction 1 (1.7 kg). Subsequently, 15 kg of toluene was added to the remaining aqueous layer and the whole was slowly stirred and heated to 70° C. After the temperature reached 70° C., the solution was stirred for 30 minutes and then allowed to stand for 4 hours. Thereafter, as in the case of the sample 6, the collection of the toluene layer, concentration, dehydration, crystallization with hexane, and drying were performed to collect a fraction 2 (0.9 kg).

The obtained each sample was subjected to measurement by GPC as in Example 1. As a result of determination of the peak areas of the diol compound and methoxypolyethylene glycol as in Example 1, the respective amounts of the high-molecular-weight impurity in the fractions 1 to 2 were 1.08% and 1.24%.

### Example 9

In a 200 mL four-neck flask fitted with a mechanical stirring apparatus, a Dimroth condenser, a thermometer, and a nitrogen-introducing tube were placed 10 g of α-t-butoxy-polyethylene glycol (molecular weight: 40,000, amount of high-molecular-weight impurity: 6.08%), 66.5 g of toluene, and 3.5 g of ethanol, and the whole was slowly stirred and heated to 69° C. After the temperature reached 69° C., the solution was stirred for 30 minutes and allowed to stand for 10 minutes. The organic layer as the separated upper layer was collected in a 300 mL eggplant-shape flask using a pipette. The organic layer containing toluene as a main component was concentrated at 80° C. to 20 g on an evaporator and, after the concentrate was cooled to 25° C. with stirring using a magnetic stirrer, 20 g of hexane was added thereto to precipitate crystals. The slurry was stirred for 30 minutes and filtrated and, after the residue was washed with 20 g of hexane, drying was performed under vacuum to collect a fraction 1 (0.8 g). Subsequently, 66.5 g of toluene and 3.5 g of ethanol were added to the remaining aqueous layer and the whole was slowly stirred and heated to 70° C. After the temperature reached 70° C., the solution was stirred for 30 minutes and then allowed to stand for 30 minutes. Thereafter, as in the case of the fraction 1, the collection of the toluene layer, concentration, crystallization with hexane, and drying were performed to collect a fraction 2 (3.0 g).

The amounts of the high-molecular-weight impurity in the obtained fractions 1 to 2 were 0.96% and 0.16%, respectively.

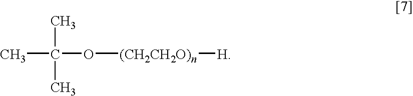

The polyethylene glycol impurity to be removed in the following Example 10 is an impurity originated from a polyethylene glycol compound having an about one-half molecular weight whose molecular weight is lower than that of the objective compound, which is mainly generated by decomposition in the reaction process of derivatization.

### Example 10

In a 300 mL four-neck flask fitted with a mechanical stirring apparatus, a Dimroth condenser, a thermometer, and a nitrogen-introducing tube were placed 10 g of a branched polyethylene glycol represented by the formula [8] (molecular weight: 40,000, amount of low-molecular-weight impurity: 2.36%) and 100 g of toluene, which were then dissolved at 50° C. under nitrogen with stirring using a mantle heater. Thereto was added 100 g of a 10% by mass aqueous sodium chloride solution, and the whole was slowly stirred and heated to 68° C. After the temperature reached 68° C., the solution was stirred for 30 minutes and, after stopping the stirring, was allowed to stand at the same time for 30 minutes to effect layer separation. The organic layer as the separated upper layer was collected in a 300 mL eggplant-shape flask using a pipette. The organic layer containing toluene as a main component was concentrated at 80° C. to 20 g on an evaporator and, after the concentrate was cooled to 25° C. with stirring using a magnetic stirrer, 20 g of hexane was added thereto to precipitate crystals. The slurry was stirred for 30 minutes and filtrated and, after the residue was washed with 20 g of hexane, drying was performed under vacuum to collect a fraction 1 (3.0 g). Subsequently, 100 g of toluene was added to the remaining aqueous layer and the whole was slowly stirred and heated to 68° C. After the temperature reached 68° C., the solution was stirred for 30 minutes and then allowed to stand for 30 minutes. Thereafter, as in the case of the fraction 1, the collection of the toluene layer, concentration, crystallization with hexane, and drying were performed to collect a fraction 2 (1.0 g). In the following, similar operations as in the case of the fraction 2 were repeated to collect a fraction 3 (1.5 g). Moreover, 100 g of toluene was added to the aqueous layer on which the treatment for the fraction 3 was finished, and the whole was stirred at 70° C. for 20 minutes and allowed to stand for 20 minutes, followed by performing concentration, dissolution into 20 g of ethyl acetate, crystallization with hexane, and drying to collect a fraction 4 (1.2 g).

The amounts of the low-molecular-weight impurity in the obtained fractions 1 to 4 were 5.36%, 4.17%, 1.59% and 0.00%.

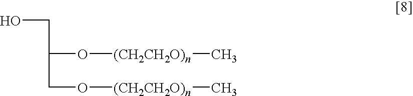

The polyethylene glycol impurity to be removed in the following Example 11 is an impurity originated from the diol compound having a molecular weight of about 4,000 whose molecular weight is lower than that of the objective compound, which is attributable to water mixed into a branched polyethylene glycol having a molecular weight of 40,000 and represented by the formula [8] used as an starting material for polymerization in the synthesis of a branched polyethylene glycol represented by the formula [9].

### Example 11

In a 300 mL four-neck flask fitted with a mechanical stirring apparatus, a Dimroth condenser, a thermometer, and a nitrogen-introducing tube were placed 10 g of a branched polyethylene glycol represented by the formula [9] (molecular weight: 42,000, n′=about 45, amount of low-molecular-weight impurity: 2.55%) and 100 g of toluene, which were then dissolved at 50° C. under nitrogen with stirring using a mantle heater. Thereto was added 100 g of a 10% by mass aqueous sodium chloride solution, and the whole was slowly stirred and heated to 67° C. After the temperature reached 67° C., the solution was stirred for 30 minutes and, after stopping the stirring, was allowed to stand at the same time for 30 minutes to effect layer separation. The organic layer as the separated upper layer was collected in a 300 mL eggplant-shape flask using a pipette. The organic layer containing toluene as a main component was concentrated at 80° C. to 20 g on an evaporator and, after the concentrate was cooled to 25° C. with stirring using a magnetic stirrer, 20 g of hexane was added thereto to precipitate crystals. The slurry was stirred for 30 minutes and filtrated and, after the residue was washed with 20 g of hexane, drying was performed under vacuum to collect a fraction 1 (4.2 g). Subsequently, 100 g of toluene was added to the remaining aqueous layer and the whole was slowly stirred and heated to 70° C. After the temperature reached 70° C., the solution was stirred for 30 minutes and then allowed to stand for 30 minutes. Thereafter, as in the case of the fraction 1, the collection of the toluene layer, concentration, crystallization with hexane, and drying were performed to collect a fraction 2 (3.8 g).

The amounts of the low-molecular-weight impurity in the obtained fractions 1 to 2 were 5.02% and 0.14%.

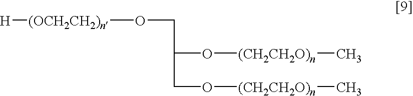

TABLE 1

| | | | | | Amount of | | | | | |
|---|---|---|---|---|---|---|---|---|---|---|
| | | | Organic | | solvent | | | Extraction | Impurity | |
| | | Molecular | Solvent | Salt | (organic | Initial | Fraction | temperature | in | |
| Example | Structure | weight | component | concentration | solvent:water) | impurity % | No. | (° C.) | fraction % | Yield % |
| 1 | MeO-PEG-H [4] | 30000 | toluene | 10 wt % | 10:10 | 2.81 | 1 | 68 | 0.39 | 27 |
| | | | | | | | 2 | 68 | 0.46 | 24 |
| | | | | | | | 3 | 68 | 0.55 | 20 |
| | | | | | | | 4 | 68 | 1.65 | 10 |
| 2 | MeO-PEG-H [4] | 40000 | toluene | 10 wt % | 10:5 | 2.80 | 1 | 68 | 0.42 | 20 |
| | | | | | | | 2 | 68 | 0.17 | 10 |
| | | | | | | | 3 | 68 | 0.55 | 10 |
| 3 | MeO-PEG-H [4] | 40000 | toluene | 10 wt % | 3:3 | 2.80 | 1 | 68 | 0.68 | 35 |
| | | | | | | | 2 | 68 | 0.37 | 18 |
| | | | | | | | 3 | 68 | 0.39 | 8 |
| 4 | MeO-PEG-H [4] | 30000 | toluene/ | 15 wt % | 5:5 | 2.81 | 1 | 53 | 0.46 | 10 |
| | | | ethyl acetate | | | | 2 | 55 | 2.08 | 66 |
| | | | (5/5) | | | | | | | |
| 5 | Diethoxy | 30000 | Ethyl | 13 wt % | 5:5 | 3.26 | 1 | 54 | 0.33 | 25 |
| | propanoxide- | | acetate | | | | | | | |
| | PEG-Me [5] | | | | | | | | | |

TABLE 2

| | | | | | Amount of | | | | | |
|---|---|---|---|---|---|---|---|---|---|---|
| | | | Organic | | solvent | | | Extraction | Impurity | |
| | | Molecular | solvent | Salt | (organic | Initial | Fraction | temperature | in | |
| Example | Structure | weight | component | concentration | solvent:water) | impurity % | No. | (° C.) | fraction % | Yield % |
| 6 | Benzyloxy- | 30000 | toluene | 10 wt % | 7:7 | 3.29 | 1 | 68 | 2.74 | 12 |
| | PEG-H | | toluene/ethanol | | | | 2 | 69 | 1.86 | 26 |
| | [6] | | (95/5) | | | | 3 | 69 | 1.01 | 21 |
| | | | | | | | 4 | 69 | 0.38 | 12 |
| 7 | MeO-PEG-H | 40000 | toluene | 10 wt % | 5:5 | 2.80 | 1 | 68 | 1.01 | 54 |
| | [4] | | | | | | 2 | 68 | 0.58 | 12 |
| 8 | MeO-PEG-H | 40000 | toluene | 10 wt % | 4:5 | 2.80 | 1 | 70 | 1.08 | 34 |
| | [4] | | | | | | 2 | 70 | 1.24 | 18 |
| 9 | t-BuO-PEG-H | 40000 | toluene/ethanol | 10 wt % | 7:7 | 6.08 | 1 | 69 | 0.96 | 8 |
| | [4] | | (95/5) | | | | 2 | 70 | 0.16 | 30 |
| 10 | HO- | 40000 | toluene | 10 wt % | 10:10 | 2.36 | 1 | 68 | 5.36 | 30 |
| | Glycerine- | | | | | | 2 | 68 | 4.17 | 10 |
| | (PEG-M)2 [8] | | | | | | 3 | 68 | 1.59 | 15 |
| | | | | | | | 4 | 70 | 0.00 | 33 |
| 11 | HO-PEG- | 42000 | toluene | 10 wt % | 5:5 | 2.55 | 1 | 67 | 5.02 | 42 |
| | Glycerine- | | | | | | 2 | 70 | 0.14 | 38 |
| | (PEG-M)2 [9] | | | | | | | | | |

1. A purification method of a high-molecular-weight polyethylene glycol compound through removing, from a high-molecular-weight polyethylene glycol compound whose total average number of moles of ethylene oxide units added in the molecule is 220 to 4500, a polyethylene glycol impurity different in molecular weight, which comprises:
(A) a mixing step of, in a state that the high-molecular-weight polyethylene glycol compound is dissolved in at least one of water and one or more organic solvents selected from the group consisting of aromatic hydrocarbon solvents having 8 or less carbon atoms in total and ester compound solvents having 5 or less carbon atoms in total, mixing the water and the one or more organic solvents; and
(B) a separation step of separating the resulting mixture into an organic layer and an aqueous layer and separating the organic layer from the aqueous layer.

2. The method according to claim 1, wherein the high-molecular-weight polyethylene glycol compound is represented by the general formula [1]: 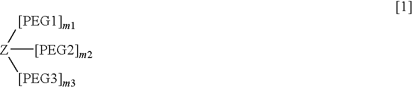
wherein Z is a divalent to octavalent bonding site having 30 or less atoms in total excluding hydrogen atom(s); PEG1, PEG2, and PEG3 are polyethylene glycol chains each having a different structure containing a bonding site and a terminal group from one another, and PEG1 and PEG2 are linear ones and PEG3 is branched one, respectively; m1, m2, and m3 represent the numbers of PEG1, PEG2, and PEG3 which bond to Z, respectively; and 0≦m1≦8, 0≦m2≦8, 0≦m3≦8, and 2≦m1+m2+m3≦8.

3. The method according to claim 1, wherein an organic solvent is newly added to the aqueous layer separated in the separation step (B), and the mixing step (A) and the separation step (B) are repeated.

4. The method according to claim 1, wherein water is newly added to the organic layer separated in the separation step (B), and the mixing step (A) and the separation step (B) are repeated.

5. The method according to claim 1, wherein the organic solvent is one or more solvents selected from the group consisting of xylene, toluene, benzene, methyl acetate, ethyl acetate, and butyl acetate.

6. The method according to claim 5, wherein the organic solvent is toluene or ethyl acetate.

7. The method according to claim 1, wherein one or more additive solvents selected from the group consisting of hexane, cyclohexane, methylene chloride, chloroform, methanol, ethanol, isopropanol, tert-butanol, diethyl ether, methyl tert-butyl ether, tetrahydrofuran, dimethyl sulfoxide, N,N′-dimethylform sulfoxide, and N,N′-dimethylacetamide are mixed into the organic solvent in an amount of 10% by mass.

8. The method according to claim 7, wherein the additive solvent is one or more solvents selected from the group consisting of methanol and ethanol.

9. The method according to claim 1, wherein at least one of an organic salt and an inorganic salt is dissolved into the water.

10. The method according to claim 9, wherein 3 to 20% by mass of an alkali metal inorganic salt or an alkali metal organic salt is dissolved into the water.

11. The method according to claim 1, wherein the mixing step (A) and the separation step (B) are carried out at 50 to 90° C.

12. The method according to claim 1, wherein the mass of the organic solvent is 1 to 50 mass times the mass of the high-molecular-weight polyethylene glycol compound and the mass of water or the total mass of the water, the organic salt, and the inorganic salt is 0.1 to 50 mass times the mass of the high-molecular-weight polyethylene glycol compound.

13. The method according to claim 1, wherein the mass of the high-molecular-weight polyethylene glycol compound is 2 to 50 when the total mass of the organic solvent(s) and the water at the time of mixing is regarded as 100.

14. The method according to claim 1, wherein the total average number of moles of ethylene oxide units added in the molecule of the high-molecular-weight polyethylene glycol compound is 440 to 3500.

15. The method according to claim 2, wherein, in the general formula [1], m1 is 1, m2 is 1, and m3 is 0; and PEG1 is represented by the following general formula [2] and PEG2 is represented by the following general formula [3]:
—(CH₂CH₂O)ₙ₁-(A¹)ₐ-R¹ [2]
—(CH₂CH₂O)ₙ₂-(A²)_{b}-X² [3]
wherein R¹ is a hydrocarbon group having 1 to 7 carbon atoms or an acetal group having 4 to 9 carbon atoms; X² is a functional group or a protective group of a functional group and is different from R¹; n1 and n2 each is the average number of moles of ethylene oxide units added and n1+n2 is 220 or more and 4500 or less; A¹ and A² each independently is a divalent bonding site group having 30 or less carbon atoms and consisting of —CH₂—, —O—, —S—, —NH—, —CONH—, —NHCO—, —OCONH—, —NHOCO—, —COO—, —OCO—, —COS—, —SOC—, —S—S—, and a combination of groups selected from the group consisting of them, which does not contain —CH₂CH₂—O—; and a and b are the numbers of units of A¹ and A², respectively and each is 0 or 1.

16. The method according to claim 15, wherein Z is —O—, X² is a hydroxyl group, a is 0, b is 1, and A² is —CH₂—CH₂—.

17. The method according to claim 15, wherein R¹ is a methyl group.

18. The method according to claim 2, wherein, in the general formula [1], m1 is 2 or more and 7 or less, m2 is 1, and m3 is 0; and PEG1 is represented by the following general formula [2] and PEG2 is represented by the following general formula [3]:
—(CH₂CH₂O)ₙ₁-(A¹)ₐ-R¹ [2]
—(CH₂CH₂O)ₙ₂-(A²)_{b}—X² [3]
wherein R¹ is a hydrocarbon group having 1 to 7 carbon atoms or a functional group or a protective group of a functional group; X² is a functional group or a protective group of a functional group and is different from R¹; n1 and n2 each is the average number of moles of ethylene oxide units added and (n1×m1)+n2 is 220 or more and 4500 or less; A¹ and A² each independently is a divalent bonding site group having 30 or less carbon atoms and consisting of —CH₂—, —O—, —S—, —NH—, —CONH—, —NHCO—, —OCONH—, —NHOCO—, —COO—, —OCO—, —COS—, —SOC—, —S—S—, and a combination of groups selected from the group consisting of them, which does not contain —CH₂CH₂—O—; and a and b are the numbers of units of A¹ and A², respectively and each is 0 or 1.

19. The method according to claim 2, wherein, in the general formula [1], m1 is 0, m2 is 1, and m3 is 2 or more and 7 or less; and PEG2 is represented by the following general formula [3] and PEG3 is represented by the following general formula [4]:
—(CH₂CH₂O)ₙ₂-(A²)_{b}—X² [3]
—(CH₂CH₂O)ₙ₃—Z′—[(CH₂CH₂O)ₙ₄-(A³)_{c}—R³]m₄ [4]
wherein R³ is a hydrocarbon group having 1 to 7 carbon atoms or a functional group or a protective group of a functional group; X² is a functional group or a protective group of a functional group and is different from R³; n2, n3, and n4 each is the average number of moles of ethylene oxide units added and n2+(n3+(n4×m4))×m3 is 220 or more and 4500 or less; A² and A³ each independently is a divalent bonding site group having 30 or less atoms in total excluding hydrogen atom(s) and consisting of —CH₂—, —O—, —S—, —NH—, —CONH—, —NHCO—, —OCONH—, —NHOCO—, —COO—, —OCO—, —COS—, —SOC—, —S—S—, and a combination of groups selected from the group consisting of them, which does not contain —CH₂CH₂—O—; Z′ is a divalent to nonavalent bonding site having 30 or less carbon atoms; m4 is the number of [(CH₂CH₂O)ₙ₄-(A³)_{c}-R³] bonded to Z′ and m4 is 1 or more and 8 or less; and b and c are the numbers of units of A² and A³, respectively and each is 0 or 1.

20. The method according to claim 1, wherein the high-molecular-weight polyethylene glycol compound is collected from the aqueous layer.

21. The method according to claim 1, wherein the high-molecular-weight polyethylene glycol compound is collected from the organic layer.
